(19) United States
(12) Patent Application Publication
(10) Pub. No.: US 2011/0270135 A1
(43) Pub. Date: Nov. 3, 2011

(54) Augmented reality for testing and training of human performance

(76) Inventors: Christopher John Dooley, Medina, OH (US); Barry James French, Bay Village, OH (US)

(21) Appl. No.: 12/927,943

(22) Filed: Nov. 30, 2010

Related U.S. Application Data
(60) Provisional application No. 61/283,156, filed on Nov. 30, 2009.

(51) Int. Cl. A61B 5/103

(52) U.S. Cl. 600/595

(57) ABSTRACT

A system for continuously monitoring a user's motion and for continuously providing realtime visual physical performance information to the user while the user is moving to enable the user to detect physical performance constructs that expose the user to increased risk of injury or that reduce the user's physical performance. The system includes multiple passive controllers 100A-F for measuring the user's motion, a computing device 102 for communicating with wearable display glasses 120 and the passive controllers 100A-F to provide realtime physical performance feedback to the user. The computing device 102 also transmits physical performance constructs to the wearable display glasses 120 to enable the user to determine if his or her movement can cause injury or reduce physical performance.

This Application is based on Provisional Application No. ______, filed Nov. 30, 2009 by Barry James French.

### FIELD OF INVENTION

The present invention assesses factors relating to a user's kinematics and/or physical performance during locomotion, and for providing visual stimuli (cuing) and continuous real-time feedback relating to the user's physical performance and/or kinematics regardless of the direction in which the user is moving or the direction at which the user is gazing (looking). It is estimated that at least 80% of the information an athlete relies on during game play is obtained visually.

### BACKGROUND OF THE PRIOR ART

For the purposes of this application the terms “user” or “athlete” will apply to persons using the present invention regardless of their interests, abilities and/or objectives. The present invention has applications that include, but are not limited to, healthcare/rehabilitation, fitness, performance enhancement/athlete development, sports, dance, martial arts and entertainment.

For the purposes of this application, the term “kinematics” will be used in reference to those factors reflective of the athlete's “form” (posture or stance) whether in a static position or while moving, as well as how these factors may be material to the athlete's physical performance and susceptibility to sports-related injury. The sciences that study these factors include:

Biomechanics—the physics of human motion. The study of the forces produced by and acting on the body. There are three terms associated with biomechanics: kinematics, kinetics, and kinesiology.

Kinematics—the temporal and spatial characteristics of motion.

Kinetics—forces that act upon, cause, modify, facilitate, or inhibit motion

Kinesiology—the science of motion. It can be termed applied functional anatomy.

The term “kinetic chain” refers to the body and its extremities, consisting of bony segments linked by a series of joints. The kinetic chain concept likens these segments and their linkages to a chain.

Sports physicians, therapists, trainers and coaches generally agree that it is the athlete with superior abilities to react, accelerate, decelerate and abruptly change direction while under control who excels in competition and is less likely to be injured. Reaction-based sports such as football, basketball and soccer, challenge the athlete to respond adeptly to the unpredictable and to move confidently in all vector directions. This unpredictable nature of sports competition is one factor that exposes the athlete to injury, notably lower extremity injuries. In sport competition, the athlete must draw from a repertoire of sensory-motor skills which includes balance and postural control, stability and the ability to anticipate competitor responses, the ability to generate and control powerful, rapid, coordinated movements of the entire body, and reaction times and anticipation that exceed those of the opponent. The quality of the athlete's stance (posture) during movement is one modifiable factor that impacts both performance and safety.

Key components of effective and safe movement include the athlete's stance, footwork, acceleration and deceleration capabilities, and the degree to which there is effective control of the body core especially during braking, cutting and landing maneuvers. The depth of the athlete's stance is just one determinant of an effective athletic stance. Athletes may be left vulnerable to the intrinsic challenges of dealing with the unpredictable nature of competition if their training is unrealistic or devoid of the means to assess key factors relating to both their physical performance and kinematics. Accordingly, testing and training programs that create a more accurate analog of the types of movement challenges inherent in actual sports competition may be more beneficial than testing and/or training that relies on drills delivering predictable (planned) challenges to the athlete.

Research confirms that serious, season-ending knee injuries are epidemic in sport. A lack of effective movement training and inefficient biomechanics can predispose athletes to non-contact knee injuries. The International Olympic Committee reported that “almost 80% of ACL injuries are non-contact . . . (and that) injuries often occur when landing from a jump, cutting or decelerating.”' Renstrom, P., Ljungqvist, A., Arendt, E., Beynnon, B., Fukubayashi, T., Garrett, W., et al. (2008). Non-contact ACL injuries in female athletes: an International Olympic Committee current concepts statement.

The practice of movement strategies that lead to more effective kinematics may be more productive by the delivery of timely, sensitive and relevant feedback relating to the athlete's kinematics and physical performance during each phase of movement, specifically when the athlete is changing direction, braking (decelerating), landing, accelerating, rotating, etc. Whether landing from a jump, cutting or decelerating, avoiding excessive dynamic valgus of the knee (“knock-kneed” knee position) and landing or braking straight-legged (knees in extension) can reduce the risk for ACL knee injuries. Studies have suggested that women tend to land with less knee flexion (bending), and in general, maintain a straighter knee during game play than do their male counterparts. Particularly troublesome are hard landings or braking with valgus when the knee is near extension. Athletes that hold their knees straighter (“extension”) upon landing from a jump or braking action (“deceleration”) increase the forces on the knee joint. Executing cutting maneuvers from a more erect position may also increase the risk of a knee injury. Learning to bend at the knees and hips, i.e., to assume a deeper stance, can reduce the stress on the knees by enabling the muscular system to act as a shock absorber.

Programs designed to reduce the incidence of lower extremity injuries, specifically knee and ankle injuries, are often devoid of means for making key measurements relating to the athlete's physical performance and kinematics. They are also devoid of means for providing essentially continuous, realtime visual feedback when the athlete is moving in various vector directions or is gazing (looking) in a variety of directions, i.e. when the athlete's viewpoint is constantly changing.

There is a growing consensus among clinicians that movement training that corrects improper kinematics may reduce the exposure that athletes have to knee and ankle injuries. Teaching of correct mechanics for sports-specific movement is an important step for an athlete striving to reach his or her genetic potential, to preventing injuries, or to fully restore mobility after an injury. Yet few athlete development programs or coaches apparently teach the mechanics of integrating and coordinating the actions of the entire body during sport-specific movement. Nor do they have the means of actually quantifying the degree to which the athlete is successful in coordinating the actions of her body while rapidly changing directions, decelerating or landing. Accordingly, there is a well-documented need for programs that prevent sports injuries as well as improve performance.

Movements can be executed with power, balance and precision when the entire body works effectively together. There must be a coordinated, properly sequenced (timed) involvement of the muscles of the torso and the extremities. During vigorous movement such as rapid changes in direction, the muscles of the trunk undergo a series of contractions to give optimum support to the extremities. Power originates from the rotation of the trunk that begins with the contraction of the lower abdominal muscles which diffuses to the trunk and upper extremities to facilitate the movement of the torso around the central-vertical axis of the body. Synchronization of lower extremity movement with the concerted action of the torso and arms further increases the stabilization of the core and lower extremities. Essentially, properly timed firing of certain upper extremity muscle groups appears to provide a base of support from which the athlete can more adeptly accelerate and decelerate. By contrast, a relaxed upper body during explosive movements may act to “absorb” energy generated from the core and lower extremities rather than effectively transmitting it. Teaching the athlete how to effectively involve the upper and lower extremities so that they work in concert may result in almost immediate improvements in the athlete's explosive movements with an accompanying reduced risk of injury. This holds true whether the athlete is locomoting or is engaged in an episodic event such as swinging a baseball bat or golf club.

Martial arts is exemplary of a physical endeavor that emphasizes the refinement of the martial artist's form (efficient kinematics) to maximize the generation of power via the coordination of the athlete's entire kinetic chain. This perfection of movement acts to maximize balance, agility, stability, quickness, reactions, and power, as well as the ability to withstand impact to the body. Coordinating the actions of the extremities with the athlete's body core is material to maximizing performance and reducing the risk of injury. Another physical endeavor that emphasizes the refinement of 3-dimensional movement is ballet as well as other forms of dance. Ballerinas/dancers strive to perfect their movement abilities to maximize the grace, beauty and power of their movement.

A research paper supported by the IOC speculated that consistent (regular) training may be necessary for long-term meaningful results from injury prevention programs, stating that “Maintenance and compliance of prevention programmes before, during and after the sports participation season are essential to mimimise injuries.” Accordingly, it is believed that a testing and training program (modality) that is game-like and interactive may act to improve compliance with the training prescription.

There is a wide continuum of performance enhancement/athlete development and rehabilitation programs designed to develop the performance capacities required for reaction-based sports. Programs vary based on the degree to which they incorporate technology to deliver both planned and unplanned stimuli, and to which they measure and provide feedback relating to the essential components of physical performance and/or athlete kinematics. At one end of the continuum of training approaches are traditional drills and programs that employ low tech means. These programs may employ speed and agility drills that typically prescribe a movement path where the distance and direction to be traveled by the athlete are known in advance. Such drills typically do not deliver to the athlete unpredictable, interactive cues. Plus traditional tests and drills are typically limited to the use of a stopwatch to measure the elapsed time to complete the pre-planned course. Examples of such drills include strategically placed ground-mounted cones, ladders and sprint training over a known distance and direction. The athlete begins such drills by responding to a whistle, or verbal command. While helpful for initial training stages, pre-planned training activities are less challenging than spontaneous cues that train the athlete's ability to sense changes in the environment, decide the proper action, react and then rapidly execute while maintaining proper body mechanics. As mentioned above, it is estimated that at least 80% of the information an athlete relies on during game play is obtained visually; lending further support for training regimens that challenge the athlete's ability to sense, process and react as well as execute.

Elapsed time measured by a stopwatch can serve to compare one athlete to another, or compare one athlete's performance over time, but more than the measure of elapsed time is needed to either test or optimize each critical component of physical performance or the athlete's kinematics. As British scientist Lord Kelvin stated, “If you can't measure it, you can't improve it.” For example, to maximize the athlete's ability to react to sport-specific cues, the athlete or coach benefits from having the means to actually measure the athlete's reaction time, which a stop watch cannot practically do. The same applies to other components of physical performance such as the ability to accelerate, decelerate or measure the depth of the athlete's stance during actual movement. The more granular (detailed) and immediate the information transmitted to the athlete, the more effective the management of the athlete's training program can be managed.

It is especially valuable to deliver to the athlete essentially realtime visual feedback when she is in a stage of locomotion (movement) that is associated with a heightened risk of injury. As discussed above, examples of such precarious stages of movement include landing from a jump, braking and aggressive cutting. Physicians, exercise physiologists, coaches, biomechanists and physical therapists and other professionals related disciplines are knowledgeable of what constitutes the types of movement that expose the athlete to increased risk of injury.

At the other end of the continuum are technology-based solutions. “Sport simulators” are exemplary of this category, as they address many of the identified deficits of conventional athlete development programming by creating an interactive testing and training experience for the athlete. They also have measurement capabilities that did not previously exist with conventional performance enhancement/athlete development programs. Since actual game play creates different neuromuscular and musculoskeletal stresses than pre-planned drills do, sports simulators deliver both planned and unplanned sport-specific cues to the athlete. With high-speed tracking of athlete movement, sports simulators can measure and report such performance factors in multiple vectors as:
Reaction Time—The elapsed time from the presentation of a visual cue to the initiation of the correct movement response
Acceleration—The measurement of the athlete's 1st step quickness
Deceleration—The measurement of the athlete's ability to brake
Velocity—The measurement of the athlete's speed
Cutting/Agility—The continuous tracking of the athlete's body core to measure the accelerations, velocity and decelerations associated with the movement phases relating to changes-in-direction (cutting)
Stance—The measurement of the depth of the athlete's stance during movement to determine the depth of stance that maximizes agility and improves safety

These measures and other measurements not discussed above enable a more accurate and sensitive gauge of physical performance capabilities; information that can be used with performance enhancement/injury prevention programs to estimate the athlete's propensity for future injury and to improve sport-specific performance. Exemplary of such devices include: French et. al. U.S. Pat. No. 5,469,740 teaches an interactive sports simulator that employs a multiplicity of ground mounted polymeric force-sensing platforms to measure core performance capacities as the athlete moves in response to the simulator's interactive, game-like cues. Though the system was incapable of continuously tracking the athlete, as the athlete's movement was only measured when the athlete was actually in contact with one or more force platforms, it represented a meaningful step toward addressing the identified deficits of traditional drills and protocols. Certain key capabilities were now measurable, and the athlete trained by responding to interactive planned and unplanned cues, activities and games. French et. al. U.S. Pat. No. 6,308,565 B1 teaches an interactive sports simulator (trade named “TRAZER”) that tracks the athlete continuously in 3-dimensions using optical tracking means. It too delivered interactive testing and training drills, protocols and games that more closely replicated the challenges of actual sports competition. This invention has expanded measurement capabilities as a result of the ability to continuously track the movement of the athlete.

However, for certain applications, sports simulators have several inherent limitations. For example, sport simulators are typically relegated to indoor training either because natural (outdoor) sunlight may interfere with certain types of movement tracking systems (for example, optical systems are susceptible to interference from direct natural sunlight) or the simulator's visual display may appear “washed out” in direct natural sunlight. Additionally, indoor training surfaces often differ from competitive playing surfaces. For certain types of athletic movement, such as lateral movement (when the athlete moves parallel to the simulator's visual display) or linear movement (when the athlete moves toward the display screen and backpedals away from the display screen) the athlete predominately remains in continuous visual contact with the visual display, and thereby is able to view the visual presentation of real-time feedback as well as the interactive stimuli. However, this may not be the case when the athlete executes maneuvers frequently employed in reaction-based sports that cause her to turn away from the screen, and therefore lose visual contact. Representative of maneuvers include: a football linebacker dropping back into pass coverage, or a basketball player getting into position to protect the net.

Continuous tracking in a reliable and accurate manner of certain body segments of the athlete may be compromised by the known sports simulators. For example, a sport simulator employing optical tracking requires optical line-of-sight for the body part(s) being tracked. Should the athlete rotate, turn, twist or similar, such line-of-sight may be occluded, and therefore the simulator may momentarily lose tracking. During such maneuvers, the profile of the athlete as “seen” (sensed) by the tracking means may render reliable, continuous tracking of the user's knees, ankles and/or hip region, difficult or even impossible at times. Even 3D cameras measuring depth that are capable of simultaneously tracking dozens of points of the human body may not be capable of reliably tracking certain points on the athlete's body continuously.

As research has demonstrated, certain phases of locomotion are inherently more dangerous for the athlete. Coincidentally, these more risky phases of movement are often also points in time when the athlete is most likely to lose visual contact with the visual display, including maneuvers such as landings, cutting, rotating and braking. This momentary loss of “coaching cues” during the most critical phases of movement dampens the value of the athlete's training program. Therefore, the known sport simulators may not represent optimal means of assessing and/or training the athlete's kinematics during certain critical phases of movement when correction of the athlete's kinematics may have the greatest benefit.

For athletes participating in reaction-based sports involving 3-dimensional movement, as well as for certain other users, there is an identified need for a system that addresses the aforementioned deficits.

### BRIEF SUMMARY OF THE INVENTION

Some of the objectives of the present invention include: providing means for the athlete to continuously view, in essentially realtime, visual feedback that relates to the athlete's kinematics (form) during locomotion regardless of the direction in which she is moving or the direction in which he/she is looking, providing tracking means for continuously tracking during movement at least one portion of the athlete's body regardless of the direction in which he/she is moving, presenting to the athlete visual feedback (information) relating to her physical performance derived from said tracking means. Performance information may be presented in engineering units and may include, but is not limited to: reaction time, acceleration, speed, velocity, power, caloric expenditures and/or vertical changes, alternatively, visual feedback (“constructs”) can be presented in the form of game-like scores that may include, but are not limited to: game points earned, tackles, catches, blocks, touchdowns, goals or baskets scored, etc. provided such game-like feedback is directly related to the athlete's physical performance and/or kinematics.

Performance constructs employ performance information to discern certain kinematic or biomechanical factors directly relating to the athlete's safety and ability to perform. Performance parameters include, but are not limited to, the quality of the athlete's stance, i.e. the width and depth of stance, the orientation of the knees, etc., and well as the timing and magnitude of the motion of the athlete's kinetic chain. Performance parameters are material to safety and success in both real world game play, as well as in the present invention's virtual world competitions, drills, protocols and games.

These aforementioned objectives can be achieved by the use of augmented reality (“AR”). The present invention's use of AR enables the delivery of essentially continuous realtime visual feedback relating to the athlete's physical performance and/or the athlete's kinematics regardless of the vector direction in which the athlete is transiting or the direction to which the athlete is gazing (her viewpoint). With the present invention, the athlete wears a suitable Head-Mounted-Display (“HMD”); examples of suitable HMDs include optical see-through and video see-through HMDs. HMDs can also be referred to as “wearable displays.” Simply stated, AR augments reality. It superimposes digital information on top of the athlete's real world (natural) view of his/her surrounding environment. AR may also add sound and haptics to the real world view. Noted AR researcher Ron Azuma defines AR as “a technology which: (1) combines real and virtual imagery, (2) is interactive in real time, (3) registers the virtual imagery with the real world.” Unlike the previously discussed sports simulators and virtual reality, AR provides both a real-world view and a view of overlaid computer-generated graphics. This graphical overlay serves to provide visual stimuli (cuing) and visual feedback relating to the athlete's physical performance and the athlete's kinematics (form) during locomotion.

One significant advantage of AR is that it enables visual feedback to be delivered regardless of the direction in which the athlete is looking (gazing) or the vector direction to which the athlete is moving. The athlete can turn, twist, rotate and abruptly change direction to assume an alternative movement path and still benefit from visual feedback relating to her kinematics and/or physical performance. This unique capability is material to the present invention's ability to improve physical performance and/or prevent sports injuries, as it is known that athletes suffer an increased risk of lower extremity sports injuries when executing athletic maneuvers involving cutting actions, rotating, braking, landing from a jump; actions that often change the athlete's direction of gaze, or viewpoint.

The use of a head mounted display (“HMD”) used in augmented reality substitutes for the fixed mounted visual display customarily employed with known sport simulators, thereby adding flexibility to the types of environments in which the athlete may train. Predicated on the type of motion tracking system employed, with AR, the present invention may be practiced indoors or outdoors, on most game or practice surfaces, and with the potential for varying sizes of training areas. The athlete is able to move in all directions without loss of visual stimuli or feedback. The graphical overlay could take many forms. For example, static virtual object(s) could be “placed” in the real-world view at perceived locations and distances replicating a traditional cone drill, such as is frequently used to test the agility of athletes. Upon viewing the virtual cones, the athlete could initiate movement within the real world physical space to that perceived physical location where a virtual (graphic) cone or cones have been overlaid on the real-world view. In this example, the virtual cone(s) define a predictable or unpredictable movement path for the athlete, which, by way of example, could be comprised of combinations of lateral, linear and/or vertical directions. When a virtual cone is “impacted” by the athlete, which is defined by that position in real space that the athlete now occupies and that coincides with where the virtual cone has been overlaid, visual, aural or tactile feedback may be provided to the athlete.

Alternatively, dynamic (virtual) object(s) could be introduced onto the real-world view at desired locations. These dynamic objects could be imbued with certain defined purposes. For example, these dynamic virtual objects may appear be responsive to the athlete's movement, or their role may be to simply cue or lead the athlete to execute a desired movement response. The dynamic virtual object(s) could be employed to create a more realistic, sport specific training experience than those delivered by virtual static object(s). One or more dynamic virtual objects could represent opposing players in a particular sport such as football, basketball, soccer, baseball or alike. For example, the dynamic virtual object could be a football running back, with the athlete assuming the role of a football linebacker whose objective is to “tackle” the virtual linebacker by moving to the position in real space that corresponds with the perceived position of the virtual running back. In this example, the athlete's physical prowess (physical performance), sport-specific kinematics (as determined by the body-worn sensors) and his ability to “read” and anticipate the actions of the virtual running back all could assist in determining his success at tackling his virtual opponent. For example, the athlete may move sufficiently quickly to the correct field position to make the tackle, but his tackling form (kinematics) may place him/her in a less than optimal biomechanical position to efficiently stop his/her virtual opponent. Feedback could be in the form of engineering units relating to physical performance or game-like points that relate to the athlete's physical prowess and/or his/her ability to “read” the actions of his virtual opponent.

Making the virtual opponents interactive as described above may more precisely replicate the stresses inherent in actual sports competition; stresses that may place the athlete at increased risk of injury due to the need to respond instantly without prior planning. Unplanned cues act to train the athlete's ability to sense and adeptly process sport-relevant information. AR, in combination with suitable tracking means, can provide valuable information relating to the athlete's kinematics (“form”). For example, a computationally simple virtual representation of the athlete (a “stick figure” or “avatar”) could serve as a model or virtual coach or fitness or dance instructor. This avatar could either serve as a visual template for what is believed to be correct movement form or could act to visually represent (reflect or mirror) the athlete's currently measured form so as to provide realtime visual feedback; for example, certain measured aspects of the athlete's movement, such as the width and/or depth of the athlete's stance. This is especially valuable during moments when the athlete is turning, rotating, cutting or similar; phases of movement where the athlete may be most susceptible to injury.

Alternatively, the virtual instructor could lead the athlete through a training or fitness program while providing coaching tips relating to the quality of the athlete's movement or her degree of compliance with correct form. As the athlete benefits from this quality and quantification of feedback, he/she should become more comfortable moving in a more efficient stance, with the expectation of improvements in his/her agility, power, balance and stamina, while reducing unnecessary energy expenditures accompanied by a reduction in the risk of injuries.

The present invention is scalable by expanding the number of points on the athlete's body that are tracked. It should be noted that suitable means of tracking may involve the affixing of sensors at desired locations on the athlete's body, or by some tracking means located remote from the athlete's body (without affixing sensors on the athlete's body), that may alternatively be employed. Examples of such remote tracking means include camera-based tracking systems capable of tracking multiple points on the human body. Exemplary of such systems is Microsoft's Kinect product.

In the preferred embodiment, the desired tracking means comprises one or more sensors capable of sensing 6 degrees-of-freedom, and affixed to one or more points on the athlete's body. Assuming the sensors attached in the vicinity of each knee are capable of measuring 3-axes of linear motion (accelerometers) and 3-axes of rotation/orientation (gyroscopes), the present invention measures orientations as well as accelerations and position of the athlete's knees. The minimal sensor (tracking) configuration requires a sensor affixed in proximity of the athlete's head so that information relating to the head's orientation and position may be reported to the HMD. The information derived from this head-mounted sensor can also be employed to measure qualities related to the athlete's physical performance. Certain commercially available HMD have built in tracking sensors; for example, the Vuzix WRAP 920AR is a head tracking system with reportedly multiple 3-axis gyros, accelerometers and magnetoresistive sensors that provide positioning and movement tracking for yaw, pitch, roll, X, Y and Z.

An HMD with the aforementioned tracking capability can provide information regarding the athlete's performance. An additional sensor may be affixed in the area of the athlete's body core so that measurements relating to movement of the athlete's body core can be made. Such measurements may include, but are not limited to, reaction time, acceleration, velocity, deceleration, core elevation and vertical changes and estimated caloric expenditure. Such measurements can be made for each vector direction that the athlete transits; this enables comparison of performance in multi-vectors to detect deficits in the athlete's ability to move with symmetry. If a suitable heart rate sensor is worn by the athlete, heart rate could be reported as well.

The preferred embodiment includes affixing one sensor in proximity of each knee so as to determine the moment-to-moment spatial relationship of the athlete's knees, and by extension, to infer information relating to the athlete's lower chain. Affixing the sensors at other locations on the lower extremities, for example, the ankle region, does not deviate from the spirit of the present invention. The spatial relationship of the knees; their orientation, distance of separation, magnitude and timing of accelerations/decelerations associated with movement and certain other factors are prime factors relating to skilled, purposeful and safe movement. In addition to the sensor(s) affixed in proximity of the athlete's body core, sensors can be affixed to the athlete's upper extremities to provide information relating to the timing and magnitude of accelerations and/or positional changes associated with the upper extremities, alone or in combination with the body core, which can then be compared to the onset and magnitude of accelerations generated from lower extremity activity. The totality of this information relates directly to the athlete's global body performance and kinematics, and can contribute to developing and managing efficacious programs for injury prevention, rehabilitation and performance enhancement. This global performance assessment is relative to sport-specific activities that range from making a tackle to guarding an opponent in basketball.

With the aforementioned sensor tracking configuration, the following provides examples of the measurements that can be made that are relevant to the athlete's kinematics and physical performance: Width of stance is the distance separating the athlete's knees in both static positions and while the athlete is under locomotion. Width of stance is material to athlete performance and safety. And having knowledge of the spatial relationship of the athlete's knees, i.e., which foot is forward and the direction in which each knee is pointing also contributes relevant information. Stance/dynamic posture is determined by 3 factors: 1. distance separating the knees, 2. relative position of each knee in space (spatial relationship—i.e., which knee is forward) and 3. the direction each knee is pointing (orientation). Depth of stance during sport-specific movement is material to the athlete's balance and postural control, stability, and the ability to safely generate and control powerful, rapid and coordinated movements. Relationship of the knees is determined by measuring such parameters as the relative angle of the knees, which can be the basis for determining neutral, varus or valgus (“kissing knees”) knee position. Knowing this relationship may assist in determining whether, during movement, the athlete's knees remain in a neutral position or if undue valgus knee motion is observed. Having knowledge of acceleration and deceleration as they relate to the accelerations and decelerations of the body worn sensors provides further information. For sensors affixed in the vicinity of the athlete's knees, accelerations may be employed to measure the accelerations associated with the driving (push off) leg, as well as the deceleration of the braking leg, measured via the user's movement in various vector directions. Reaction Time is determined by the onset of accelerations of one or more body worn sensors in response to a cue delivered by the present invention. Reaction time and subsequent accelerations may provide data relating to the timing and magnitude of upper and lower body movement that contribute to athlete locomotion. Deceleration forces—may determine, for example, if the athlete sufficiently dampens the forces of braking with proper flexion of the knees and hips. Change in direction is recognized by several distinct phases: 1. onset of acceleration, generated by the pushing (propelling) leg, 2. deceleration of the front (braking) leg, and 3. bi-lateral change of direction. This complex action is comprised of the deceleration phase, uniform (predictable) changes in the spatial relationship of knees, and then the re-acceleration phase. Braking and landing is determined by 4 factors: 1. distance separating the knees (width of stance), 2. direction each knee is pointing (orientation), 3. angle of the knee (is the knee in proper flexion), and 4. deceleration forces. Velocity is the speed of the athlete in a given direction.

It should be noted that attaching the sensors in the region of the ankles or upper leg(s) does not deviate from the spirit of the invention, as much of the aforementioned information would still be available.

Another example of the utility of the present invention is a simple interactive reaction drill. With this drill, the athlete is presented with unpredictable visual cues that prompt him/her to move aggressively to follow the desired movement path. The timing and magnitude of the accelerations generated from the HMD tracker can be employed to measure how the athlete's head responds to the delivered cue. If a sensor is affixed to the athlete's body core as well as sensors in the vicinity of each knee, this “simple” reaction drill can assess multiple factors relating to both sport-specific performance and athlete kinematics: The elapsed time from the presentation of the visual cue to the athlete's initial movement (response) can be measured for each affixed sensor. These elapsed times provide information regarding the responsiveness of the athlete's entire kinetic chain, and the timing and magnitude of the accelerations associated with each affixed sensor provides information related to the athlete's overall kinematics and physical performance.

The present invention's “Jump” protocol is illustrative of the utility of a drill/protocol that does not involve rotating or cutting but rather training for safe and effective landings. It is designed to identify increased risk of knee injuries, to train proper landing techniques and improve the athlete's jumping ability. Assuming the sensors attached in the vicinity of each knee are capable of measuring 3-axes of linear motion and 3-axes of rotation/orientation, the present invention measures orientations as well as accelerations and position of the athlete's knees. Therefore the distance between the athlete's knees can be continuously measured to assess the width of the athlete's stance in essentially real-time. With the sensor affixed on the athlete's body core, the depth of the athlete's stance can also be measured.

The orientation of the athlete's knees and the depth of stance during sport-specific movement is material to the athlete's balance and postural control, stability, and the ability to safely generate and control powerful, rapid and coordinated movements. AR can provide the athlete continual realtime visual feedback regarding the aforementioned kinematic and physical performance factors. Accordingly, he/she can refine/modify his/her stance before jumping and upon landing as a result of realtime immediate feedback (biofeedback) that acts to reinforce proper mechanics. This example protocol begins with the user starting on an elevated platform. The athlete is instructed to jump to the floor and immediately jump as high as possible. Key parameters that may directly impact athlete safety and performance include:

Relationship of the Knees—upon landing are the athlete's knees in a neutral position or do they land in a valgus knee (“kissing knees”) position. Acceleration—to what degree does the athlete explode upward upon landing; what is his/her ability to generate power. Deceleration—does the athlete land “softly’, i.e. are the forces of landing dampened with proper flexion of the knees and hips. Width of stance—is the athlete's base of support stable, so that balance and agility are maximized. Additionally, the height of jump and depth of the athlete's landing are measured.

The present invention has the capability of assessing both movement form (”technical execution” or kinematics) and performance factors (acceleration, deceleration, velocity, power, etc.). The result is a tool to break down in real-time the complex kinematics and physical performance factors into divisible components. Several interrelated divisible components of locomotion are measured to detect athlete movement patterns that negatively impact performance or expose the athlete to an increased risk of injury. Some of the components of locomotion include, but are not limited to, the spatial relationship of the athlete's knees and the depth of his/her stance. This capability enables real-time feedback relating to a number of kinematic and physical performance factors as the athlete responds to either unplanned or planned movement challenges.

Both the training and testing aspects of the present invention benefit from AR. Beginning with simple, easily performed reactive movement tasks, the present invention's pre-established programs can vary the intensity and complexity of the athlete's reaction-based movement activities. The athlete's compliance with established operating limits can determine the rate at which she can be progressed. The device can provide individualized protocols accompanied by aural, tactile or visual feedback when training.

### BRIEF DESCRIPTION OF THE DRAWINGS

FIG. 1 depicts a system for continuously monitoring a user's motion and or continuously providing realtime visual physical performance information to the user while the user is moving; the system being illustrated by a “stickman” with passive controllers disposed upon head, arms and legs for sensing motion and for communicating to an electronics pack, which transmits physical performance information to wearable display glasses and to a base station computer that further processes performance information for feedback to the electronics pack and ultimately the wearable display glasses where the processed performance information is displayed in real time in accordance with the present invention.

FIG. 2 is a block diagram of the electronics pack of FIG. 1 having wireless connections to the passive controllers and wearable display glasses on the stickman, and to the computer.

FIG. 3 depicts an alternative embodiment of the system of FIG. 1 via the stickman wearing the same passive controllers and wearable display glasses, but wearing an electronics pack that does not communicate with a base station computer.

FIG. 4 is a block diagram of the electronics pack of FIG. 3.

FIG. 5 is a flow chart of the system of FIG. 1 in accordance with the present invention.

FIG. 6 is a flow chart of the alternative embodiment of the system of FIG. 3 in accordance with the present invention.

FIG. 7 depicts an optical overlay-based augmented reality system. Depicted here is a cluster of three trees in a real world landscape. The viewer sees the landscape as a unit when she looks through the glasses with both eyes.

FIG. 8 is a flowchart of the base station computer of FIG. 1.

### DETAILED DESCRIPTION OF THE INVENTION

The present invention is capable of identifying kinematic and/or performance factors that may expose the athlete to an increased risk of injury or that negatively impact the athlete's physical performance capabilities. Real-time visual feedback can alert the athlete to potentially dangerous, or at least potentially inefficient, movement patterns. Alternatively, the present invention can be used as an entertaining physical activity for members of the general populous, including children, seniors, patients and fitness buffs.

In summary, the present invention has the following capabilities: 1. the assessment of certain factors pertinent to the athlete's kinematics and physical performance during movement, 2. provision for real-time visual feedback regardless of the direction in which that the athlete is gazing (viewpoint) or the direction in which the athlete is moving, 3. the option of either pre-programmed testing and/or training protocols and workouts or user-determined activities, 4. multiplayer training and games.

The preferred embodiment has both solo and multi-player operational modes: User Directed Mode—this mode allows the athlete to determine the types of movements to undertake while receiving selected feedback that may include coaching tips and performance feedback. With this mode, the athlete may elect to introduce interactivity and spontaneity by having a real world training partner interact with her in the same physical space. Or the athlete can elect to receive feedback or coaching tips while training with a conventional cone drill. The selected feedback may include reaction time, 1st step quickness, depth of stance, velocity, caloric expenditure, etc. that would not be measurable with a stopwatch measuring only elapsed time. Device Program Mode—this mode has the device delivering pre-programmed training protocols to which the athlete responds and receives selected feedback. Single User Mode—both the aforementioned modes are single player modes. Multiplayer Mode—this mode provides for two-way, realtime interaction between two or more athletes within the same physical space. The objective of multiplayer activities is to introduce interactivity and spontaneity for more realistic training. The ever-changing spatial relationship between the athletes creates a competitive or cooperative experience that can realistically approximate actual game play in reaction-based sports.

To follow are examples of games and activities applicable to both single and multiplayer modes. In a single player mode, the athlete competes against a virtual opponent displayed on the HMD; in the multiplayer mode, the athlete competes with one or more real world opponents that are viewable on the HMD. Examples include: “Guard”—where the objective for the athlete is to maintain a synchronous relationship (to follow the movement path) with her real or virtual opponent. “Evade”—where the objective for the athlete is to create a brief asynchronous event (to break away) in an effort to “score” on her real or virtual opponent. “React”—where the objective for the athlete is to quickly respond (react) to a real or virtual opponent.“Mimic”—where the objective for the athlete is to mimic the movement of a real or virtual opponent that may include, for example, fitness, dance, martial arts or sport-type movement patterns.

The prime objective of the present invention is to “monitor” the athlete during locomotion in order to detect kinematic or physical performance factors that may expose the athlete to an increased risk of injury or that negatively impact the athlete's performance capabilities. At such times as the athlete's kinematics and/or physical performance are maintained within predefined acceptable limits, the athlete can be rewarded with positive feedback. However, at such times when the athlete's movement exceeds the pre-established acceptable limits, cautionary feedback may be delivered to the athlete. Certain performance ranges can be established or adjusted based on the athlete's anthropometrics, age, medical history, sport of interest, fitness level, etc. By way of example, the present invention may be programmed for “acceptable ranges” relating to the preferred depth of the athlete's stance. For example, a desired depth of stance for a certain athlete may be in a range from minus 8 inches to minus 14 inches as measured from her standing height. Feedback in the form of coaching tips (advice) can also be delivered. The feedback may be aural, tactile and/or by visual or other suitable means.

Another example of the present invention's protocol is an “agility drill.” This protocol can be used to identify athletes at increased risk of severe knee injuries and test or train the athletes' performance capabilities. As previously discussed, the present invention can also be employed in conjunction with conventional agility tests. For example, the athlete can perform a conventional cone drill designed to test the athlete's agility and quickness while the present invention delivers relevant feedback. In this example, the present invention provides additional information not provided by the conventional stopwatch, which only measures the elapsed time to complete the drill. Alternatively, the protocol elicits from the athlete multi-vector movement with visual or auditory cues that cause the athlete to move. The movement vectors can be forward, backward, side-to-side, up or down, on the diagonals or in any combination of vectors. The drills employ virtual objects to define the athlete's movement path. For example, the virtual object could be a “hurdle” that the athlete must jump over to avoid impacting a barrier.

Feedback may relate to the percentage of training time that the athlete was in compliance with a specific training parameter; for example, the percentage of training time that the athlete exhibited a proper stance. Feedback could also be as a game score that relates to the athlete's physical performance prowess, or feedback in engineering units such as heart rate, movement speed, power, acceleration, deceleration, posture, stance, etc.

The present invention also offers novel means for assessing and training of physical activities that are episodic in nature; examples of such activities include swinging a baseball bat, golf club, tennis racket, hockey stick, etc., or for throwing or striking an implement such as a baseball, football, basketball or volleyball. Numerous means are taught in the prior art for evaluating the athlete's biomechanics while engaging in such activities. Effectively swinging a sports implement involves the coordination of the athlete's entire kinetic chain in a biomechanically efficient manner. Ground-mounted force platforms and/or means for measuring the motion and/or position of strategic points on the athlete's body are the basis for determining the biomechanics (efficiency) of the athlete's swinging, throwing or hitting motion.

By way of example, U.S. Pat. No. 7,602,301 teaches the use of sensors measuring position, acceleration and orientation affixed at various points on the athlete's body to provide performance-related information and constructs related to the entire swinging motion of a golf club or baseball bat. Feedback is provided to the athlete regarding their performance upon completion of the swinging or throwing motion. However, there is no provision for providing continuous visual feedback of the athlete's kinematics or physical performance during the actual execution of the swing, throw or hit, as the rapidly changing viewpoint of the athlete as the result of head rotation makes continuous viewing of one or more stationary (fixed) monitors (displays) impractical. In fact, attempting to view a fixed display monitor while executing a technique would be counterproductive to the development of effective mechanics (form).

It is believed that the delivery of realtime visual feedback during the entire phase of a swing, throw or hit, regardless of the where the athlete is gazing, could improve the training experience. The present invention provides essentially continuous visual feedback as the athlete's head rotates through the entire swing, throw or hit phase. Body-worn sensors provide information regarding the magnitudes and and timings of accelerations of the athlete's kinetic chain during the rotational and stabilization phases of the swing. Accordingly, feedback is more immediate and therefore may be more valuable. Visual feedback may be in the form of performance constructs, such as how adeptly the athlete's stance transitions through the swing, hit or throw phase, or the timing, magnitude and coordination of the sensored points on the athlete's body.

For sports that involve an episodic event that is preceded by, and/or is followed by, aggressive locomotion, such as tennis or hockey, the present invention can provide continuous visual feedback relating to both the episodic event and the associated aggressive locomotion. For example, as the tennis player proceeds to move aggressively into a position on the court to return a volley, the present invention provides both performance information and/or performance constructs. Performance constructs may include, for example, the quality of the athlete's kinematics during braking (deceleration) and the timing and magnitude of forces along the athlete's kinetic chain as she stabilizes in preparation to hit the ball.

The present invention can also improve the assessment and training for strength and conditioning programs. A number of commercially available exergaming products or sports simulators provide visual feedback to the athlete during strength training. However, feedback is only available to the athlete at such times as the athlete is in visual contact with the display screen. This can be a deficit for exercises such as push-ups, bench press, barbell rowing and similar exercises where the athlete cannot maintain visual contact with the visual display.

The present invention can also be used by bicyclists, skiers, ice skaters and in similar sports where the performance and kinematics of each leg is material to success. For example, with a sensor affixed in the vicinity of each knee, the present invention can provide realtime information relating to the timing and acceleration of each leg. This information could reveal bilateral asymmetries or as a measure to calculate absolute power, etc. With the present invention, the athlete can receive essentially continuous feedback regarding his/her exercise form (kinematics) and her physical performance.

Three components constitute an augmented reality system: User motion tracking means, Head-Mounted Display (HMD) and body-worn computing power/capability. Feng Zhou et all identified some of the challenges of implementing AR, “(a) graphics rendering hardware and software that can create the virtual content for overlaying the real world, (b) Tracking techniques so that changes in the viewer's position can be properly reflected in the rendered graphics, (c) Tracker calibration and registration tools for precisely aligning the real and virtual views when the user view is fixed, and (d) Display hardware for merging virtual images with views of the real world.” With AR the graphic overlay is continually refreshed to reflect the movement of the athlete's head.

User motion tracking means. There are a number of suitable means that AR systems employ to track the user's moment-to-moment position. Sensing means may include a digital compass, 3-axis orientation and 3-axis accelerometers as well as differential GPS for certain outdoor applications. Additionally, passive magnetic field detection sensors can be combined with these aforementioned sensors. This use of multiple sensors generates the data to both measure and refine the user's physical performance and kinematics. For certain implementation, sensors providing only positional information, or sensors only providing orientation specific data may suffice predicated on the application.

One embodiment for tracking the user's movement is taught in US patent application US 2010/0009752 by Amir Rubin. It describes the use of multiple body-worn magnetic sensors each capable of calculating the absolute position and orientation. As taught, these sensors can be attached on a limb, the body core, or the user's head. The sensors communicate wirelessly with a “base station” through an active sensor, but the sensors can also be connected with cables to the active sensor, or all of the sensors could communicate directly with the base station wirelessly. This sensor system enables essentially the real-time tracking of the position and orientation of various points of interest on the athlete's body. Such points of interest may include one or both knees, ankles, arms, the body core and/or the user's head region. This tracking provides sufficient update rates and accuracy to effectively measure the parameters of interest to the present invention. It is immune from interference from ambient light, so it can be used outdoors. And being wireless, it does not restrict the user's movement.

Head Mounted Displays. Head-mounted displays (HMDs) enable the user to view graphics and text produced by the augmented reality system. Examples of HMD include: 1. Optical see-through, and 2. Video see-through. For the type of dynamic movement contemplated by the present invention, “optical see-through” models have certain performance benefits. Optical see-through HMDs enable the user to see the real world in addition to the graphic overlay with his natural eyes, which is preferred for the sport-specific applications of the present invention where the user may occasionally move at high speed. The HMD superimposes digital information upon the athlete's view of the training space, thereby enabling the continuous delivery of digital information regardless of the viewpoint of the athlete. With computer graphics being overlaid on the natural (real) world view, these HMD have low time delays, the athlete's view of the natural world is not degraded.

An example of an optical see-through wearable display is the Microvision Color Eyewear. It is characterized as a “retinal display”. Microvision's eyewear “combine(s) the tiny, thin PicoP full color laser projection module with . . . clear optics that channel the laser light and direct it to the viewer's eye—all without sacrificing an unobstructed view of the surroundings.” This model does not incorporate sensing means, and Microvision's retinal display is not currently in commercial production.

Video see-through HMDs use cameras mounted near the user's head/eye region to take video images of the real world and feed them back to a computing system. The computing system can then take the captured images of the real world and overlay or embed the virtual objects into each frame of video to form a composite image. This new sequence of images or video is then projected back to the HMD for viewing by the user. A known deficit with video see-through HMDs is the time lag associated with capturing, processing and displaying the augmented images; all of which can cause the user to experience a delay in viewing the images. As technology improves, this delay will be become less noticeable. Until the optical see-through HMDs are readily available, the video see-through HMDs are implemented for the preferred embodiment of the current invention. An example of a video see-through eyewear is the Vuzix WRAP 920AR, an HMD that incorporates motion tracking.

Still another approach to enabling the user to see a view of the natural world combined with computer-generated graphics can be achieved by mounting a micro LCD display inside a pair of glasses, or using a micro projector to project an image onto a small screen or glasses worn by the user.

The HMD, regardless of the type, may incorporate sensing means to determine the orientation and direction/position of the user's head (eyes). Alternatively, the AR system may incorporate a discrete sensor to track where the user's head is positioned and oriented. This is needed so the correct view of the simulation can be displayed to the user to correspond to what they are looking at in the natural world.

Several documents providing requisite technical background for implementing augmented reality are incorporated herein by reference in their entirety: “Interactive 3D modeling in outdoor augmented reality worlds” by Wayne Piekarski, US 2004/0051680—“Optical See-Through Augmented Realty Modified-Scale Display”, and US 2004/0080548—“Method and Apparatus for Augmented Reality Hybrid Tracking System with Fiducial-Based Heading Correction.”

Without proper registration of the digital information, the ability of the system to measure the physical performance or kinematics of the user, or for the static and dynamic objects to realistically interact with the user may be dampened. Distinguishable objects (“markers”) placed in the physical space may play an important role to AR's performance. US 2004/0080548 teaches the use “of a plurality of at least three tracking fiducials selectively each respectively located in fixed predetermined locations in the observation space . . . ” To effectively enable the present invention combined with AR, proper means to register and precisely align the real and virtual views is advantageous.

Body-worn computing power/capability. Examples of suitable computing devices include cellular phones and audio playback devices, or the base station can be a dedicated unit designed specifically for the present invention. The portability of the computing device is an important factor, as the user will be performing vigorous exercise while receiving biofeedback.

The various sensors of the present invention communicate with the computing device, which preferred embodiment is worn/carried on the user's body. The preferred embodiment employs an Apple iPod, iTouch or iPhone. Alternatively, the various body-worn sensors may communicate with a computing device not attached to the user. For example, the sensors may communicate with a TRAZER-like system, a PC or other similar device. The computing device may also upload user data and information to send and/or receive data and information to a personal computer and/or to a remote system preferably via a network connection, such as over the Internet, which may be maintained and operated by the user or by another third party.

Because at least some portions of systems and methods according to examples of this invention may receive data from multiple users, users can compete against one another and/or otherwise compare their performance even when the users are not physically located in the same area and/or are not competing at the same time.

Data from the invention can be transferred to a processing system and/or a feedback device (audio, visual, etc.) to enable data input, storage, analysis, and or feedback on a suitable body-worn or remotely located electronic device. Software written for the body worn computing device facilitates communication with the sensors employed. Where a commercially available sensor system is employed, software is written for the computing device that takes the positional coordinates of such sensors, as well as potentially the orientation of each sensor, and generates the displayed graphics.

Since the current commercial HMD devices use a standard VGA or other video input connection (e.g. s-video), a standard video card in the computing device would output a suitable signal to generate the display. When a micro LCD is used for the HMD, additional circuitry may be needed to power and convert the data from the computing device's video output for display on the HMD. This may be true for other HMDs as well, that do not use standard video connections and protocols.

Software may also be developed to synchronize the data from the computing device to another computer and/or the internet to facilitate sharing of information or further analysis. Data may then be saved and used for comparisons to certain metrics, or compared to other users' information.

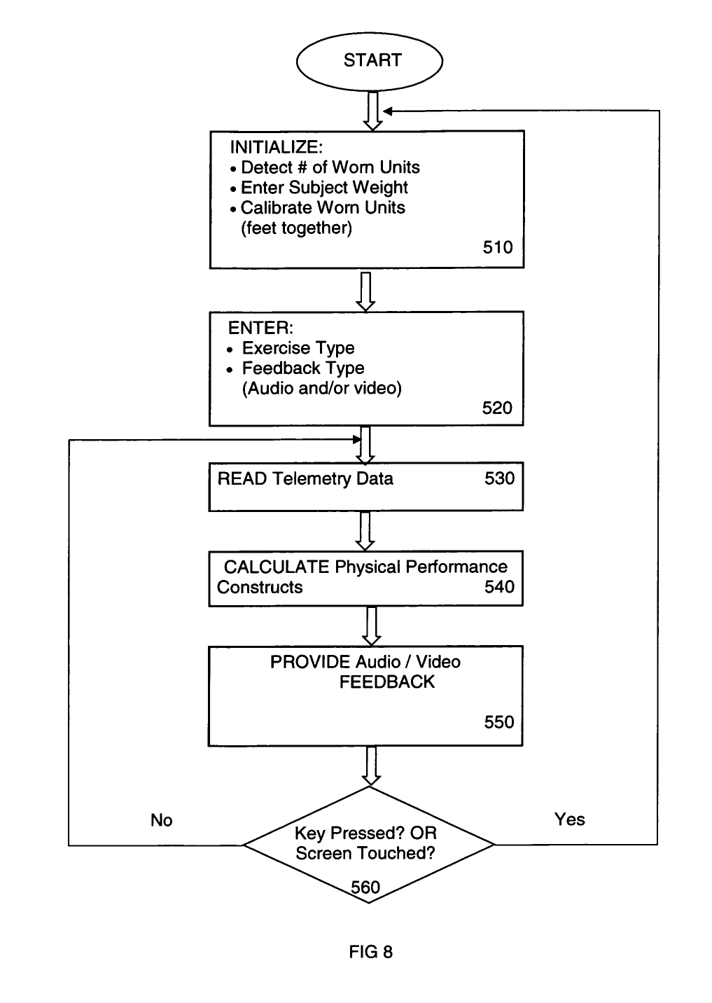

An algorithmic flowchart of the software running on the base unit is shown in FIG. 8, described below. Briefly, upon start of the algorithm, the base unit determines the number of body worn units that are within the vicinity of the device. Afterwards, it prompts the user to enter his weight, followed by a sensor calibration step where the user is instructed to stand upright with feet held together. After the completion of the initialization, the base unit enters into the operation mode, which starts with the selection of the exercise type and the preferred mode of feedback, such as audio in the form of synthesized speech and/or video in the form of bar graphs for chosen parameters. The rest of the operational mode consists of the reading telemetry data from the body worn units, the calculation of the bodily parameters using the teachings of the present invention, and the presentation of audio and/or video feedback to the user(s). This process can be interrupted by input from the user.

An advantage of the present invention is its versatility; users can test, train or play either indoors or outdoors while moving within a small or large physical space. For example, athletes training for competition can test or train on the actual competitive field of play rather than a training surface or environment. The user can perform activities (exercises or game play) requiring movement of just a few inches or many hundreds of yards or more. Regardless of the activity, the user may be provided with real-time aural, visual or tactile feedback of the user's performance and/or kinematics. This embodiment uniquely provides previously unavailable, real-time information due to the interactive nature of the drills, protocols, games and tests and the ability of the motion tracking system to track multiple points on the user's body

### DETAILED DESCRIPTION OF THE PREFERRED EMBODIMENT

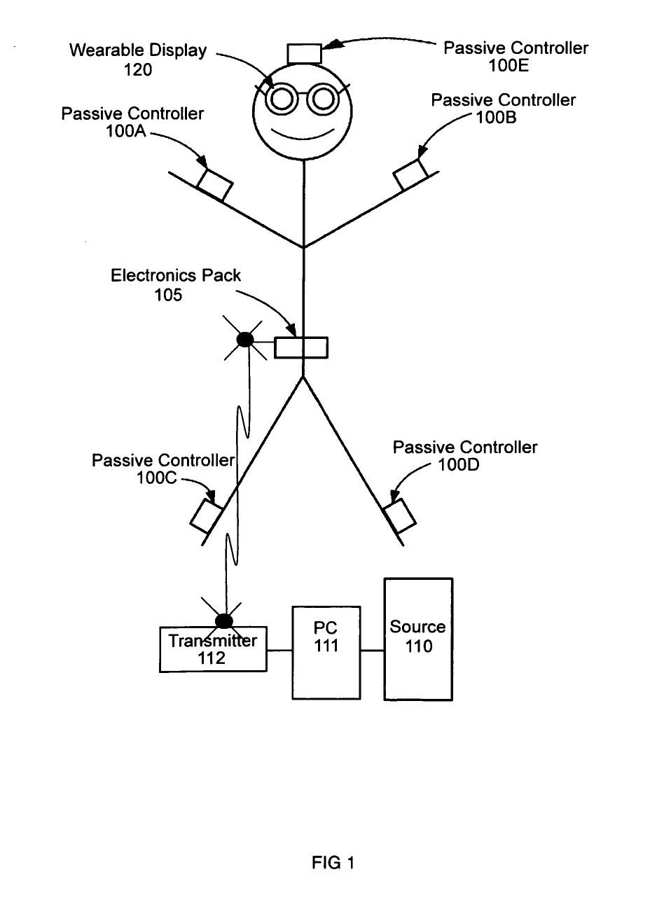

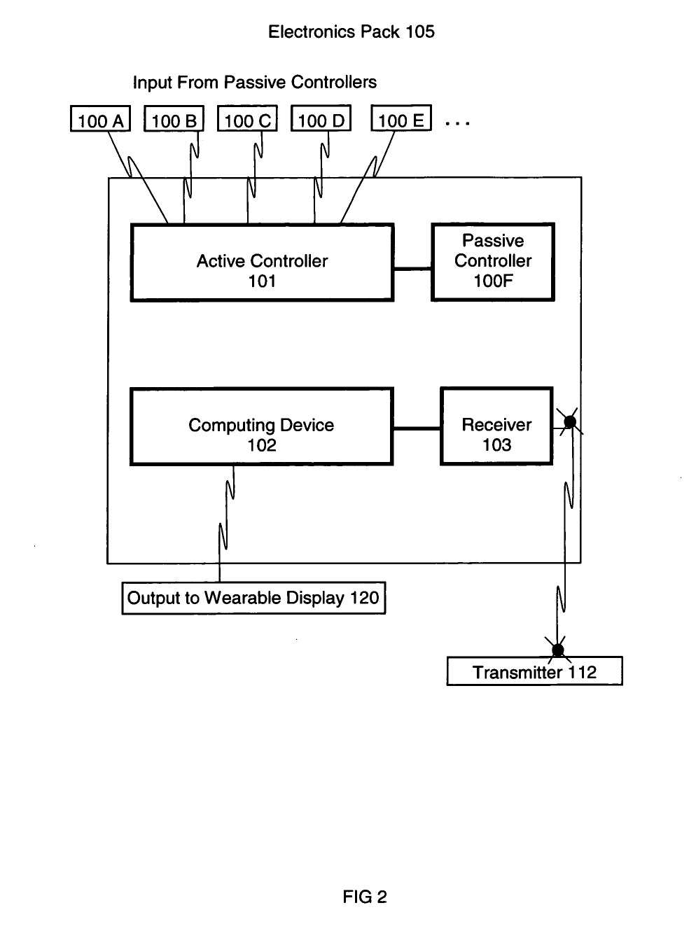

Referring now to FIGS. 1, 2 and 5, a source 110 generates a magnetic field that is detected by the passive controllers 100A-F secured to the arms, legs and head of a user as illustrated via the stickman. The passive controllers 100A-F communicate with an active controller 101 via wired or wireless transmission. The active controller 101 then communicates the position and orientation of all of the passive controllers 100A-F back to the source 110 via wireless transmission. A personal computer 111 then reads the data at the source 110 and re-transmits the data through transmitter 112 to receiver 103 wirelessly (e.g. Bluetooth, RF, etc). A body worn computing device 102 (e.g., a personal computer, smart phone, iPod, or other computing system) processes the received data and integrates the data into a running simulation. The computing device 102 is coupled via cable, or other means (preferably wireless) to a wearable display 120 for display output of the simulation in operation that includes continuously providing realtime visual physical performance information to the user while the user is moving to enable the user to detect physical performance constructs that expose the user to increased risk of injury or that reduce the user's physical performance.

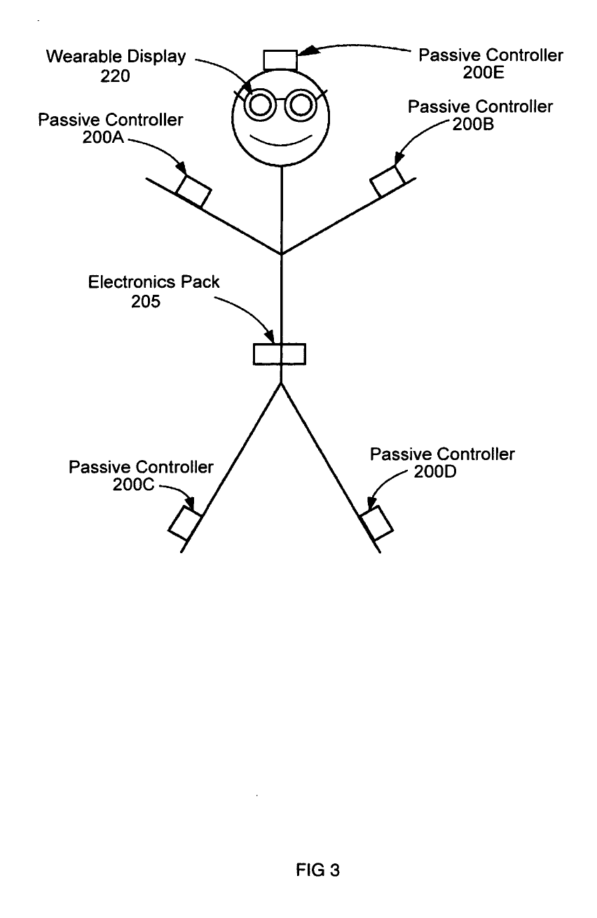

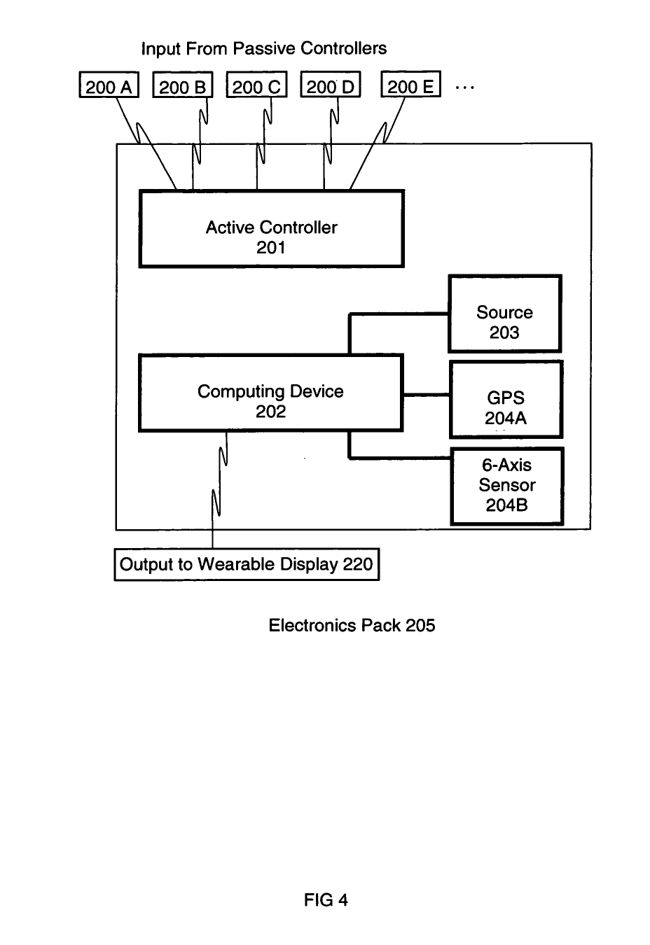

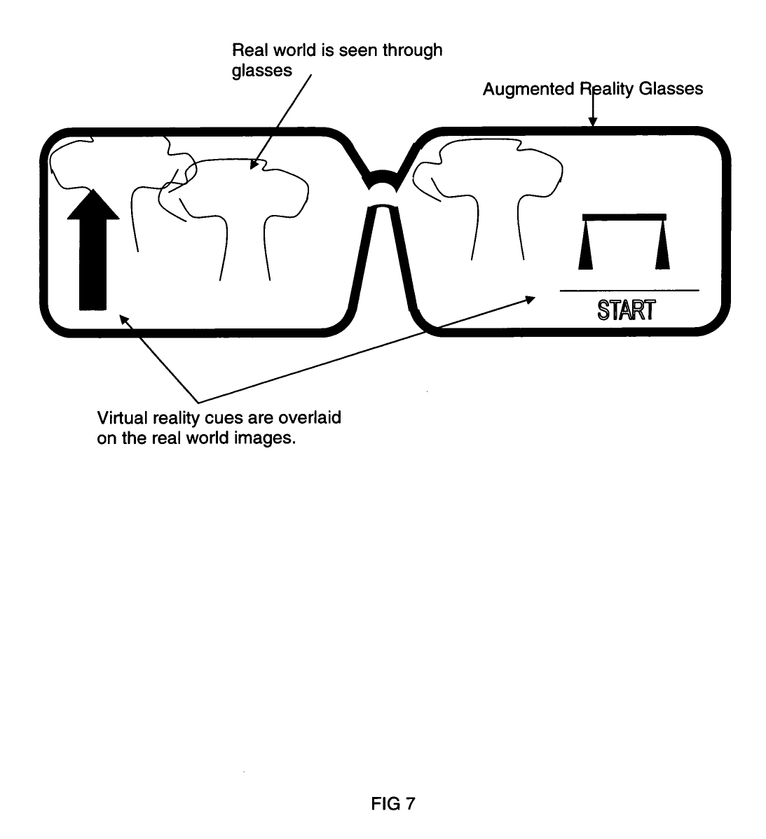

Referring now to FIGS. 3, 4 and 6, an alternative embodiment of the present invention is depicted that includes a source 203 that is body worn and generates a magnetic field which is detected by the passive controllers 200A-E. The passive controllers 200A-E communicate with an active controller 201 can via wired or wireless transmission. The active controller 201 then communicates the position and orientation of all of the passive controllers 200A-E back to the source 203 via wireless transmission. A body worn computing device 202 (e.g., a personal computer, smart phone, iPod, or other computing system) is connected to the source 203 and communicates with the source 202 via wired or wireless transmission (e.g. Bluetooth, RF, etc.). The computing device 202 is also coupled to a GPS receiver 204A or other means for determining the exact position in free space (e.g. RFID Tags, Indoor GPS, etc) and also a 6-axis sensor 204B, which contains a 3-axis accelerometer and a 3-axis gyroscope. The computing device 202 processes the received data from all three sources 203, 204A and 204B and integrates the data into the running simulation. The computing device 202 is coupled via cable, or other means to a wearable display 220 for display output of the simulation in operation that includes continuously providing realtime visual physical performance information to the user while the user is moving to enable the user to detect physical performance constructs that expose the user to increased risk of injury or that reduce the user's physical performance. Referring to FIG. 7, the wearable display 220 depicts real world images seen through the glasses 220 that include three trees, and virtual reality cues overlaid on the real world images. The virtual reality depicts a start and a racing hurdle on the right glass and an arrow on the left glass. The arrow tells the user that she must jump higher to clear the hurdle. Although the right and left glasses show different images, the user sees the three trees, hurdle and arrow as a single display.

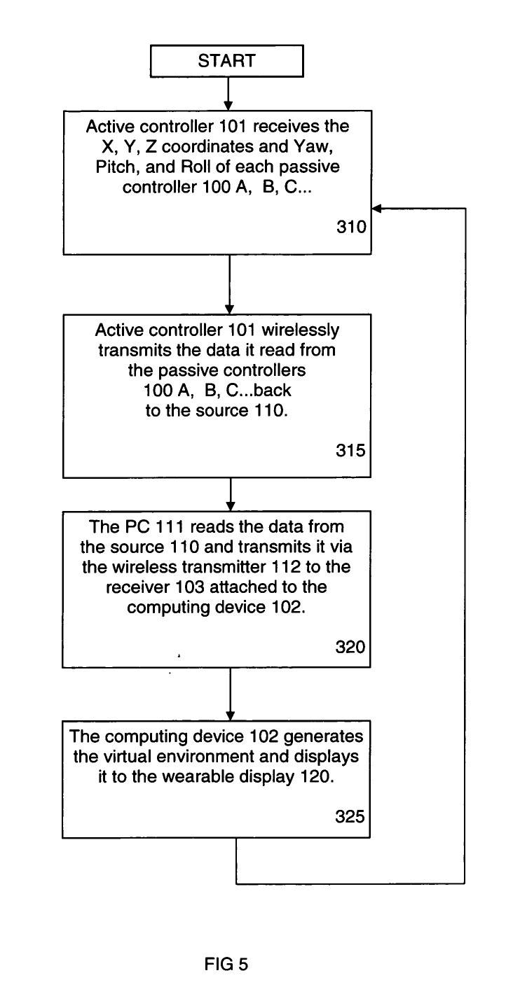

Referring now to FIG. 5, a flowchart of the preferred embodiment of the present invention can be seen. In step 310, the active controller 101 reads the X, Y, and Z locations and the Yaw, Pitch, and Roll of each passive controller 100A-F. Each of the passive controllers 100A-F is connected to the active controller 101 by wires or by a wireless communication means such as Bluetooth or RF. A suitable wireless communication device is the MotionStar Wireless LITE from Ascension Technologies. Up to 13 individual sensors can be connected to the active controller 101, which can monitor three dimensional positions and orientations of each passive controller 100A-F using a magnetic field generated from the source 110. All measurements of position and orientation are relative to the location of the source unit 110. In step 315, the active controller 101 transmits the three dimensional position and orientation of each passive controller 100A-F to the source 110 via its built in wireless transmitter.

In step 320, the personal computer 111 reads the three dimensional information from the source 110 and uses transmitter 112 to transmit the information wirelessly to receiver 103. This step is necessary because the active controller 101 transmits the data directly to the source unit 110. If the transmission protocol were known and was able to be mimicked by the body worn computing device 102, this step would not be needed, as the computing device 102 could simply communicate with the active controller 101 directly. In step 325, the computing device 102 generates the virtual simulation using the positional and orientation data from the passive controllers 100A-F and displays the information on the wearable display 120. The wearable display 120 is preferably an optical see-through HMD from Microvision, but at the current time no model is available to the public. Alternatively, a video see-through HMD from Vuzix (e.g. WRAP 920AR+) is the preferred type of HMD. Since the display obscures the user's vision, the 920AR+ contains two video cameras that record user's natural world (their viewpoint). Since this type of wearable display cannot overlay the simulation directly onto the screen, there is an additional step the computing device needs to perform. The computing device 102 needs to take the video obtained from the integrated video cameras in the wearable display 120 and combine those images with the simulation currently in progress. This combined picture of the real (natural) world plus the simulation (virtual) world can then be displayed to the user on the wearable display 120. At such time as a suitable optical see-through display is commercially available, this step will not be necessary. In an optical see-through display the wearable display is transparent and the simulation can be projected directly onto the screen and the user can see the natural world behind the display.

Some wearable displays include sensors to calculate the position and orientation of the user's head, but if not, a passive controller 100E is attached to the user's head to determine the exact position and orientation. This extra sensor allows the computing device 102 to know exactly what the user is looking at in the real and virtual worlds, so the correct camera angle of the virtual world can be displayed to correlate with the real world image the user is seeing. Without this sensor 100E, if the user turned her head to the left, the image would not change and the augmented reality simulation would not work.

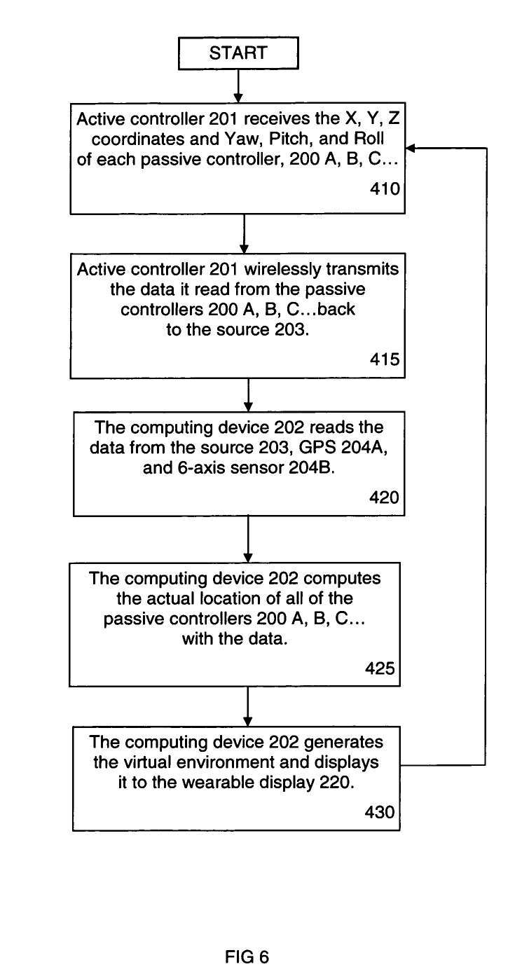

Referring now to FIG. 6, a flowchart of an alternative embodiment of the present invention can be seen. In step 410, the active controller 201 reads the X, Y, and Z locations and the Yaw, Pitch, and Roll of each passive controller 200A-E. Each of the passive controllers 200A-E is connected to the active controller 201 by wires or by a wireless communication means such as Bluetooth or RF. A suitable device as described is the MotionStar Wireless LITE from Ascension Technologies. Up to 13 individual sensors can be connected to the active controller 201, which can monitor three dimensional positions and orientations of each sensor 200A-E using a magnetic field generated from the source 203. All measurements of position and orientation are relative to the location of the source unit 203. In step 415, the active controller 201 transmits the three dimensional position and orientation of each passive controller 200A-E to the source 203 via its built in wireless transmitter.

In step 420, the body worn computing device 202 reads the three dimensional information from the source 203 and the global positional data from the GPS receiver 204A. A suitable USB GPS receiver 204A is connected to the computing device 202 via wired or other wireless transmission means. A highly accurate GPS receiver 204A is preferred as it will improve the appearance of the simulation and the accuracy of the performance data. In this embodiment the GPS receiver 204A is used to supplement the information from the passive controllers 200A-E. Since the source is now body-worn, the positional and orientation data received from the passive controllers 200A-E is now relative to the location of the source device 203. Since the GPS sensor 204A only contains the X, Y, Z positional data of itself, a means of tracking the orientation of the sensor 204A location is also needed. This is supplemented by a 6-axis sensor 204B, which can be integrated into the computing device 202 in certain instances (e.g. iPhone, iPod Touch, etc). The 6-axis sensor integrates a 3-axis accelerometer and 3-axis gyroscope. Using the integrated gyroscope, the computing device 202 now knows the exact orientation of the sensor 204B. This sensor 204B, along with the GPS sensor 204A and source 203, may be attached at the base of the spine or at other suitable positions on the body. The spine is representative of a location on the body that maintains a relatively fixed position regardless of the actions of the upper and lower body. The GPS receiver has reported accuracy to approximately 2 cm, but the frequency of GPS updates is quite small, and therefore cannot be used for a millisecond resolution position sensor. Accordingly, the GPS signal is used to correct the drift encountered when tracking a point in space by a 6-axis sensor. Since drift from the 6-axis sensor degrades over long time periods, the GPS sensor's updated position can be used to address the drift issue once a new position is known.

In some circumstances (e.g. indoors) the GPS sensor will not be able to determine the exact location of the user because the receiver cannot detect signals inside buildings. There are other positioning systems for use indoors that have accuracies in the range from an inch to a centimeter that would serve as a replacement. Indoor GPS systems as well as RFID locator systems are capable of calculating the exact position of an object indoors down to accuracies similar to those of a GPS system. The GPS sensor may be replaced by one such sensor system to facilitate the use of the invention indoors. In step 425, since the computing device 202 knows the exact orientation of the user, as well as the location of the source 203 relative to all of the passive controllers 200A-E, the computing device 202 can calculate the exact position of every passive controller 200A-E. This allows the computer 202 to place the user in the simulation properly and track the location of all sensors 200A-E over large distances. Drift encountered by the 6-axis sensor over time can be calculated out and corrected every time a new reading from the GPS signal is received. This gives the computing device 202 a millisecond resolution position and orientation of the user's current position.

In step 430 the computing device 202 generates the virtual simulation using the positional and orientation data from the sensors 200A-E and displays the information on the wearable display 220. The wearable display is preferably an optical see-through HMD from Microvision, but at the current time no model is available to the public. Instead, a video see-through HMD from Vuzix (e.g. WRAP 920AR+) is employed. Since the display obscures the user's vision, the 920AR+ contains two video cameras that record the user's natural world (his/her viewpoint). Since the wearable display 220 cannot overlay the simulation directly onto the screen, there is an extra step the computing device 202 needs to perform. The computing device 202 needs to take the video obtained from the integrated video cameras in the wearable display and combine those images with the simulation currently in progress. This combined picture of the real (natural) world plus the simulation (virtual) world can then be displayed to the user on the wearable display. This step would not be necessary with optical see-through displays. In an optical see-through display the wearable display is transparent and the simulation can be projected directly onto the screen and the user can see the natural world behind the display.

Some wearable displays include sensors to calculate the position and orientation of the user's head, but if not, a passive controller 200E is attached to the user's head to determine the exact position and orientation. This extra sensor enables the computing device to know exactly what the user is looking at in the real and virtual worlds so the correct camera angle of the virtual world can be displayed to correlate with the real world image the user is seeing. Without this sensor 200E, if the user turned her head to the left, the image would not change and the augmented reality simulation would not work. Referring now to FIG. 8, a flowchart of the computing device of FIG. 1 is depicted. Referring to block 510, the computing device 102 determines the number of body worn passive controllers 100A-F that are within the vicinity of the source 110 (block 510). The computing device 102 then prompts the user to enter his weight, followed by a sensor calibration step where the user is instructed to stand upright with feet held together (block 510). After the completion of the initialization (block 510), the computing device 102 enters into the operation mode, which starts with the selection of the exercise type and the preferred mode of feedback, such as audio in the form of synthesized speech and/or video in the form of bar graphs for chosen parameters (block 520). The computing device 102 then reads the data provided by the passive controllers 100A-F (block 530), calculates predetermined physical performance constructs (block 540), and provides realtime visual (or audio) feedback to the user via the wearable display 120 (block 550). Referring now to block 560, If the user presses a key or touches a screen, the computing device 102 then returns to block 510 and the system is reinitialized, otherwise, the computing device 102 returns to block 530 where the computing device 102 again reads the data provided by the passive controllers 100A-F to ultimately provide new physical performance constructs to the user for continuously monitoring his or her motion and for continuously providing realtime visual physical performance information to the user while the user is moving to enable the user to detect physical performance constructs that expose the user to increased risk of injury or that reduce the user's physical performance.

1. A system for continuously monitoring a user's motion and for continuously providing realtime visual physical performance information to the user while the user is moving to enable the user to detect physical performance constructs that expose the user to increased risk of injury or that reduce the user's physical performance comprising:
means for continuously measuring three axes of linear motion of predetermined body portions of the user;
means for inputting said measured three axes of linear motion into processing means;
means for determining physical performance information from said continuously measured three axes of linear motion;
means for calculating predetermined physical performance constructs from said continuously measured three axes of linear motion; and
means for continuously providing realtime visual physical performance information and realtime physical performance constructs to the user while the user is moving, whereby the user is enabled to detect physical performance constructs that expose the user to increased risk of injury and/or that reduce the user's physical performance.

2. The system of claim 1 wherein said measuring means includes at least one accelerometer secured to a predetermined body portion of the user.

3. The system of claim 1 wherein said inputting means includes a wireless transmitter.

4. The system of claim 1 wherein said inputting means includes a base-station having receiving, computer, display and download capabilities.

5. The system of claim 4 wherein said base-station is secured to the user.

6. The system of claim 4 wherein said base-station is remote to the user.

7. The system of claim 1 wherein said processing means includes a microprocessor, said microprocessor determining physical performance information and calculating predetermined physical performance constructs from said continuously measured three axes of linear motion.

8. The system of claim 1 wherein said means for calculating predetermined physical performance constructs includes the user selecting one of the following protocols:
pre-established physical performance protocols that the user ultimately follows; and
a user directed mode that allows the user to establish his or her physical performance protocols.

9. The system of claim 1 wherein means for continuously providing realtime visual physical performance information and realtime physical performance constructs to the user while the user is moving includes a head mounted display system that continuously receives in realtime physical performance information and physical performance constructs from said processing means.

10. The system of claim 1 wherein said physical performance information includes the group consisting of reaction time, acceleration, deceleration, velocity, speed, jump height, vertical changes, caloric expenditures and combinations thereof.

11. The system of claim 1 wherein said physical performance constructs include the group consisting of the width of the user's stance, orientation of the user's knees, depth of the user's stance, coordination of the user's upper extremities, coordination of the user's lower extremities, coordination of the users body core, and combinations thereof.

12. The system of claim 1 wherein said means for continuously providing realtime physical performance information and realtime physical performance constructs to the user includes the group consisting of a visual display, an audio device, a tactile device, and combinations thereof.

13. A system for assessing physical performance information relating to a user's kinematics and/or physical performance during locomotion, and for providing continuous real-time feedback relating to the user's physical performance and/or kinematics regardless of the direction the user is moving or the direction the user is looking comprising:
means for continuously tracking a user's motion;
means for inputting said user's motion into a computer;
means for continuously calculating predetermined physical performance constructs from said continuous tracking of the user's motion; and
means for continuously providing realtime physical performance information and/or realtime physical performance constructs to the user while the user is moving, whereby the user is enabled to assess physical performance and/or kinematics regardless of the direction the user is moving or the direction the user is looking.

14. A method for providing continuous, realtime physical performance information to a user while the user is moving, said method including the step of:
measuring motion of predetermined body portions of a user;
inputting said measured motion into a computer secured to the user;
calculating physical performance information from said measured motion;
transmitting said physical performance information continuously to the user in realtime; and
displaying said physical performance information continuously to the user in realtime while the user is moving, whereby the user is able to avoid physical movement that could result in injury to the user and/or that could decrease the user's performance capabilities.

15. The method of claim 14 wherein the step of measuring motion includes the step of monitoring the user during locomotion in order to maintain kinematic and/or physical performance factors within predefined limits.

16. The method of claim 14 wherein the step of displaying said physical performance information continuously to the user in realtime while the user is moving includes the step of displaying a real or virtual opponent to the user such that the user must perform at least one of the following steps:
maintaining a synchronous relationship (to follow the movement path) with said real or virtual opponent;
initiating an asynchronous (to separate from) event to evade said real or virtual opponent to ultimately score on said real or virtual opponent;
reacting to said real or virtual opponent; and
mimicking the movement of said real or virtual opponent.

17. The method of claim 16 wherein the step of mimicking said real or virtual opponent includes the step of performing at least one of the following steps:
dancing with said real or virtual opponent;
fighting with said real or virtual opponent; and
running with said real or virtual opponent.

18. The method of claim 14 wherein the step of displaying said physical performance information continuously to the user in realtime while the user is moving includes the step of providing optical see-through head mounted displays to enable the user to see the real world and the graphic overlay simultaneously.

19. The method of claim 14 wherein the step of measuring motion includes the step of using means for measuring six degrees of motion of predetermined portions the user's body.

20. The method of claim 14 wherein the step of measuring motion includes the step of providing realtime visual feedback relating to the user's physical performance and/or the athlete's kinematics regardless of the vector direction in which the athlete is transiting or the direction to which the athlete is gazing.
